(19) United States
(12) Patent Application Publication
(10) Pub. No.: US 2026/0014316 A1
(43) Pub. Date: Jan. 15, 2026

(54) INFUSION SET FOR AN INFUSION DEVICE

(71) Applicant: Ypsomed AG, Burgdorf (CH)

(72) Inventors: Rolf Marggi, Bern (CH); Andreas Zurflüh, Hasle (CH)

(21) Appl. No.: 19/338,817

(22) Filed: Sep. 24, 2025

Related U.S. Application Data
(63) Continuation of application No. PCT/EP2024/056502, filed on Mar. 12, 2024.

(30) Foreign Application Priority Data
Mar. 30, 2023 (CH) CH000355/2023

(51) Int. Cl. A61M 5/158; A61L 29/02

(52) U.S. Cl. CPC A61M 5/158; A61L 29/02; A61M 2005/1586

(57) ABSTRACT

An infusion set includes a base part for attaching to the skin of a patient. A cannula for supplying a liquid medicament into the body of the patient is provided on the distal side of the base part. Additionally, a cap part for holding the infusion set by means of the patient or an insertion device or a piercing aid is provided, a needle being provided on the distal side thereof. The base part and the cap part can be connected together in order to attach the base part connected to the cap part to the skin of the patient. The needle is arranged within the cannula, with the tip of the needle having a conical shape, and the needle is made of a solid material.

### CROSS-REFERENCE TO RELATED APPLICATIONS

This application is a continuation of International Patent Application No. PCT/EP2024/056502, filed Mar. 12, 2024, entitled “INFUSION SET FOR AN INFUSION DEVICE,” which in turn claims priority to Swiss Patent Application No. CH000355/2023, filed Mar. 30, 2023, entitled “INFUSION SET FOR AN INFUSION DEVICE”, each of which is incorporated by reference herein, in the entirety and for all purposes.

### TECHNICAL FIELD

Infusion sets for an infusion device are provided, which are used to deliver a liquid product, particularly a liquid medicament, from an infusion device into a patient's body.

### BACKGROUND

The term, “medicament,” here comprises any flowable medical formulation which is suitable for controlled administration through a means such as a cannula or hollow needle—for example, comprising a liquid, a solution, a gel, an emulsion, or a fine suspension which contains one or more medical active substances. A medicament can be a single active substance composition or a pre-mixed or co-formulated composition, having multiple active substances, from a single container. The term medicament includes drugs such as peptides (e.g., insulins, insulin-containing medicaments, GLP-1-containing and derived or analogous preparations), proteins and hormones, biologically obtained or active substances, active substances based on hormones or genes, nutrient formulations, enzymes and further substances both in solid (suspended) or liquid form but also polysaccharides, vaccines, DNS or RNS or oligonucleotides, antibodies or parts of antibodies, and suitable base, auxiliary and excipient substances.

For patients who have a regular need for liquid medicament, the required amount of medicament can be delivered to the body in liquid form using a cannula placed in the subcutaneous tissue over a longer period of time. For this purpose, an infusion set with a cannula made of a soft material or a hard material is attached to the patient's skin in such a way that the cannula penetrates through the skin into the subcutaneous tissue. The administration of the required amount of medicament in liquid form is thus carried out by an infusion device, in particular by an infusion pump, particularly by an automatic infusion pump via a supply line to the patient, wherein the cannula arranged in the infusion set supplies the patient with the liquid medicament.

Infusion sets are known from the prior art. The document WO2020/084374A1 discloses an infusion set comprising a base part with a soft cannula, wherein the cannula serves to penetrate through the skin into the subcutaneous tissue in order to administer a liquid medicament to a patient. Furthermore, the infusion set has a cap part, wherein the cap part serves to hold the infusion set by means of the patient or by means of an insertion device or piercing aid, so that the base part connected to the cap part can be attached to the patient's skin. This cap part contains a hollow needle which is arranged inside the cannula to guide the cannula.

The disadvantage of this infusion set is that it is difficult to handle.

### SUMMARY

It is an object of the present disclosure to provide an alternative infusion set, in which the handling of the infusion set is facilitated.

This object is achieved by the subject matter of the present disclosure.

In the infusion set according to implementations of the present disclosure, “in the distal direction”, means the side to which an adhesive bandage can be attached in order to attach the infusion set to the skin of a patient. In the infusion set according to implementations of the present disclosure, “in the proximal direction”, means the side that can be connected to an infusion device.

The infusion set according to implementations can be connected to an infusion device, in particular to an infusion pump, particularly to an automatic infusion pump. Infusion devices can be used for the continuous administration of infusions, particularly of medicaments in liquid form. The infusion device may be a reusable infusion device, where a new container pre-filled with a liquid medicament can be inserted into the reusable infusion device. On the other hand, the infusion device may be a single-use infusion device, where the infusion device is discarded when a container pre-filled with a liquid medicament is empty. The infusion set may include a base part for attachment to the skin of a patient, where the base part may have a proximal side and a distal side. Furthermore, the base part can have a cannula for supplying the liquid medicament into the patient's body. The cannula is designed as a hollow needle, allowing liquid medicament to be administered into the patient's body.

The cannula is particularly designed as a soft cannula. The soft cannula can be made of a soft material, in particular a polymer. The cannula can be provided on the distal side of the base part.

Furthermore, the infusion set can include a connector part with a supply line, in particular a tube. The connector part can have a proximal side and a distal side. The supply line can be used to connect to the infusion device. Furthermore, the supply line can be connected to the base part by means of a piercing cannula in order to form a fluid path between the connector part and the base part.

Furthermore, the infusion set may include a cap part for holding the infusion set by means of the patient or by means of an insertion device or piercing aid, where the cap part may have a distal side and a proximal side. The cap part can be cap-shaped.

The cap part of the infusion set includes a needle. The needle is made of solid material. The needle made of solid material has the advantage that the needle has greater flexural rigidity than a cannula with the same outer diameter, and is therefore less easily deformed.

In addition, the needle according to implementations has a tip which has a conical shape. The conical tip of the needle has the advantage that the cannula is not damaged by the tip of the needle when the needle of the cap part is pushed or inserted into the cannula of the base part during assembly of the infusion set.

The tip of the needle can be straight or obliquely conical. Particularly, the tip of the needle is straight and conical. This design has the advantage that the needle of the cap part can be pushed or inserted into the cannula of the base part particularly easily and without damaging the cannula. In addition, it can be ensured that no transverse forces act on the needle when the needle is inserted it into the skin or subcutaneous tissue, where the needle is not deformed and the cannula can be positioned more precisely in the subcutaneous tissue.

The cap part can be or is detachably connected to the base part. The connection can be designed as a snap connection. Alternative connections may be provided. The cap part can be connected, in particular snapped, to the base part in such a way that the needle can be arranged concentrically in the cannula of the base part. The cannula can be used to deliver a liquid medicament into a patient's body. The cap part can be or is located on the proximal side of the base part. The concentric arrangement of the cannula and needle can serve to guide the cannula when the cannula is inserted into the patient's skin or subcutaneous tissue. At least the distal tip of the needle can protrude beyond the distal end of the cannula, with the needle of the cap part being arranged within the cannula of the base part. The needle is arranged inside the cannula when the base part is connected to the cap part or when the base part and the cap part are in a connected state. Furthermore, an adhesive bandage can be arranged on the distal side of the base part. The adhesive bandage can be used to secure the infusion set to the patient's skin. In order to attach the infusion set to the patient's skin, the base part, which is connected to the cap part and is particularly snapped together, can be stuck to the patient's skin using an adhesive bandage, and a protective film can be removed from one adhesive side of the adhesive bandage beforehand. The cannula, which is held by the needle, can penetrate through the skin and into the patient's subcutaneous tissue.

The conical tip of the needle particularly has a cone angle (W) greater than or equal to 10° and less than or equal to 35°, between 14° and 30°, or approximately 20°. This design of the cone angle (W) ensures that when the cannula is inserted into the skin or subcutaneous tissue of the patient, with the needle arranged within the cannula and at least the distal tip of the needle projecting beyond the distal end of the cannula, the skin or subcutaneous tissue of the patient is injured as little as possible. In addition, when the needle is used according to implementations, the patient's skin is penetrated in such a way that the skin or subcutaneous tissue heals as quickly and scar-free as possible after the puncture.

In order to prevent irritation or allergic reactions of the patient's skin or subcutaneous tissue as far as possible when the needle is inserted, particularly if the distal end of the needle protrudes beyond the distal end of the cannula, the entire needle or at least part of the needle, such as the tip of the needle, is made of metal, thereby ensuring great strength of the needle. The use of stainless steel, in particular stainless steel according to ISO15510 or a biocompatible steel grade as defined by ISO10993, is particularly suitable for preventing irritation or allergic reactions upon penetration of the patient's skin or subcutaneous tissue.

Alternatively, the needle may be made from another metal including but not limited to iron, copper, bronze, aluminum, magnesium or an alloy, in particular a steel alloy. However, plastic can also be used.

At least the part of the needle that comes into contact with the skin or subcutaneous tissue should be made of the above-mentioned material in order to avoid irritation or allergic reactions of the patient's skin or subcutaneous tissue.

In order to connect the infusion set to an infusion device, the cap part with the needle can first be removed from the base part, and a connector part which can include a supply line, in particular a tube and an adapter, can be attached to the base part of the infusion set. The connection between the base part and the connector part can be designed as a snap connection. Alternative connections may be provided. A first end of the supply line can be welded and/or glued to the adapter. A second end of the supply line can be welded and/or glued to the connector part. The adapter of the connector part can be detachably attached to a corresponding connecting element of the infusion device. The adapter of the connector part can be connected to the infusion device using a bayonet lock, Luer lock or another closure. This allows a fluid path to be created from the infusion device via the infusion set to the patient.

The infusion set may have a detachable protective element. The protective element can serve to protect against puncture injuries caused by the needle of the cap part of the infusion set. The protective element can be sleeve-shaped. Alternative designs of the protective element may be provided. One or more longitudinal ribs can be provided on an outer shell surface of the protective element to improve gripping of the protective element. This allows the patient to simply remove the protective element from the base part before applying the infusion set to the patient's skin and, after removing the infusion set from the patient's skin, simply replace it over the needle of the cap part, thus protecting the patient from puncture injuries. The protective element can be detachably connected to the base part and/or the cap part with a force fit and/or a form fit in order to protect the patient from puncture injuries.

The base part of the infusion set can be flat. For guiding and/or positioning the base part relative to the cap part and/or to the connector part, a base cylinder may be provided on the proximal side of the base part of the infusion set. For this purpose, a corresponding cap cylinder and/or connector cylinder can also be provided on the cap part and/or connector part, in particular on the distal side of the cap part and/or connector part. The base cylinder can protrude from the proximal side of the base part in the proximal direction. The base cylinder can be cylindrical. The base cylinder can be arranged concentrically with the cannula of the infusion set.

Furthermore, a cannula cylinder can be provided on the distal side of the base part of the infusion set. The cannula cylinder can be cylindrical. The cannula cylinder can protrude from the distal side of the base part in the distal direction. The cannula can be secured in the corresponding cannula cylinder, in particular in a passage provided in the cannula cylinder, and protrude from the corresponding cannula cylinder in the distal direction in order to deliver the liquid medicament to the patient.

The length (L) of the needle projecting from the distal side of the base part is greater than or equal to 10 mm and less than or equal to 40 mm, between 20 mm and 30 mm, such as approximately 24.4 mm when the needle is arranged within the cannula. The length (L) of the needle is preferably measured from the distal side of the cannula cylinder of the base part to the distal end of the tip of the needle. These lengths (L) of needle are particularly suitable for a cannula of approximately 5 mm to 10 mm, measured from the distal end of the cannula cylinder. The selection of the length (L) of the needle depends on the length (L) of the cannula, where the needle is placed inside the cannula and at least the distal tip of the needle should protrude beyond the distal end of the cannula. Particularly, at least the distal tip of the needle extends beyond the distal end of the cannula. More particularly, the entire tip of the needle and at least part of a shaft of the needle extend beyond the distal end of the cannula. This arrangement ensures that the cannula can be inserted into the patient's skin or subcutaneous tissue as painlessly as possible and without causing major injuries to the skin or subcutaneous tissue.

Particularly, the diameter (D) of the needle is greater than or equal to 0.2 mm and less than or equal to 0.55 mm, between 0.26 mm and 0.5 mm, such as approximately 0.36 mm. The diameter (D) of the needle is measured at the straight shaft of the needle. This diameter (D) is particularly suitable for a cannula with a diameter (D) of 0.2 mm to 0.6 mm. The diameter (D) of the needle is matched to the diameter (D) of the cannula so that the cannula can be guided well and, in particular, inserted into the patient's skin or subcutaneous tissue painlessly and without injury.

The base part and/or the cap part of the infusion set can be circular and/or elastic. This design ensures that the base part can be attached flush and well to the patient's skin and that the cap part can be easily connected and detached from the base part. Other configurations may be provided.

Particularly, the base part and the cap part are connected to each other in a rotationally fixed manner. For this purpose, a locking tooth can be provided on the cap part, which can engage in a projection-and-recess arrangement of the base part in order to form an anti-rotation device.

When the cap part is connected to the base part, in particular snapped together, the locking tooth of the cap part can engage in the recess of the projection-and-recess arrangement of the base part to form an anti-rotation device. The anti-rotation device between the cap part and the base part allows the patient to more easily remove the protective film of the adhesive bandage from the base part. To do this, the patient grasps a grip part provided on the cap part to remove the protective film of the adhesive bandage of the base part that is snapped onto the cap part. The grip part is provided on an outer shell surface of the cap part. The grip element may extend from the outer shell surface of the cap part, particularly in the proximal direction. The grip element can hold the needle, where the needle can extend from the inner shell surface of the cap part in the distal direction. In addition, the grip element can be designed in such a way that the infusion set can be inserted more easily into an insertion device or a piercing aid. Alternatively, the grip element may be designed such that the patient can better grip the grip element in order to place or attach the base part connected to the cap part onto the patient's skin.

Particularly, the cap part can have a holding element. The holding element can be arranged on an outer shell surface of the cap part. The holding element can extend from the outer shell surface of the cap part, particularly in the proximal direction. More particularly, two parallel holding elements can be provided on the outer shell surface of the cap part. The holding element can be designed and/or arranged on the cap part in such a way that the patient can connect the cap part to the base part, in particular snap it together, and can detach the cap part from the base part.

The holding element can also serve to hold the cap part, which is connected, in particular snapped, to the base part in an insertion device or in a piercing aid. The cannula of the infusion set can be placed directly into the skin or subcutaneous tissue using the insertion device or piercing aid and held in place with the adhesive bandage. In addition, the cap part can be detached from the base part by pressing the two parallel holding elements together. The two holding elements can be pressed towards each other or pre-tensioned inwards, where the connection, in particular the snap connection between the cap part and the base part, is released.

### BRIEF DESCRIPTION OF THE DRAWINGS

Implementations will be described below with reference to a number of drawings. Both individually and in any combination, the features disclosed herein advantageously develop implementations of the present disclosure further. In the drawings:

FIG. 1 shows an exploded view of the infusion set according to implementations of the present disclosure, showing a cap part and a base part of the infusion set.

FIG. 2 shows a longitudinal sectional view of the infusion set according to FIG. 1, where the cap part is connected, in particular snapped, to the base part.

FIG. 3 shows a longitudinal sectional view of a needle of the cap part according to FIG. 1.

FIG. 4 shows an isometric view of the needle of the cap part according to FIG. 2, where the needle is arranged within the cannula of the base part, and at least a distal tip of the needle projects beyond a distal end of the cannula.

### DETAILED DESCRIPTION

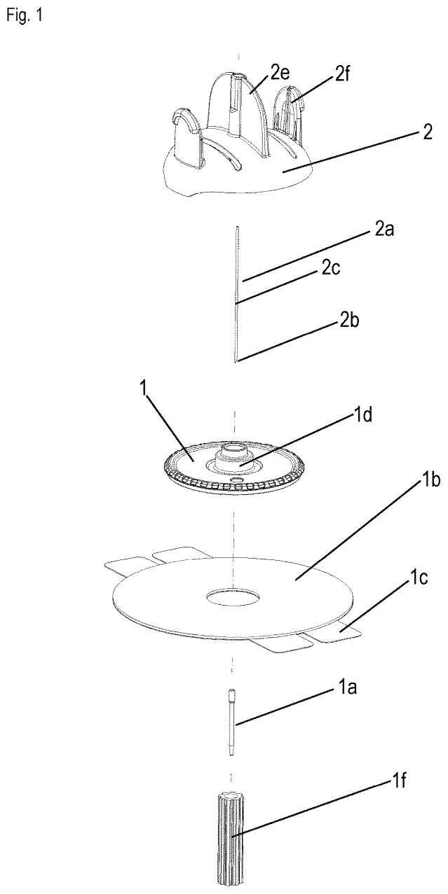

FIG. 1 shows an exploded view of an infusion set according to implementations of the present disclosure, where a cap part (2) and a base part (1) of the infusion set can be seen.

The infusion set includes a circular and cap-shaped cap part (2) with a needle (2a). The cap part (2) is elastic. The needle (2a) includes a conical tip (2b) and a shaft (2c), the needle being formed from solid material. The needle (2a) formed from solid material has the advantage that the needle (2a) has a greater flexural rigidity than a hollow needle with the same outer diameter and is therefore less easily deformed.

The needle (2a) can be made of metal, in particular of stainless steel, particularly of stainless steel according to ISO15510 or a biocompatible steel quality according to ISO10993. The use of these materials prevents irritation or allergic reactions when penetrating the patient's skin or subcutaneous tissue.

Alternatively, the needle may be made from another metal such as iron, copper, bronze, aluminum, magnesium or an alloy, in particular a steel alloy.

The needle (2a) is fixed to the cap part (2). One, in particular two holding elements (2f) and a grip part (2e) are provided on an outer shell surface of the cap part (2). The holding element (2f) can be used to hold the infusion set and the grip part (2e) can be used to insert the infusion set into an insertion device or into a piercing aid.

Furthermore, the infusion set includes a circular, in particular flat base part (1). To protect against puncture injuries caused by the needle (2a), a protective element (1f) can be provided on the base part (1). The base part further includes a base cylinder (1d) which is cylindrical in shape. The base cylinder (1d) is arranged on the proximal side of the base part (1) and protrudes in the proximal direction. A cannula (1a) is provided in the base cylinder (1d) via a passage. The cannula (1a) is designed as a soft cannula (1a) and is thus made of a soft material. On the distal side of the base part (1) there is an adhesive bandage (1b) for attaching the infusion set to the skin of a patient. The distal side of the adhesive bandage (1b) can be covered with a protective film (1c). Before applying the infusion set to the patient's skin, the patient removes the protective film (1c) from the adhesive bandage (1b).

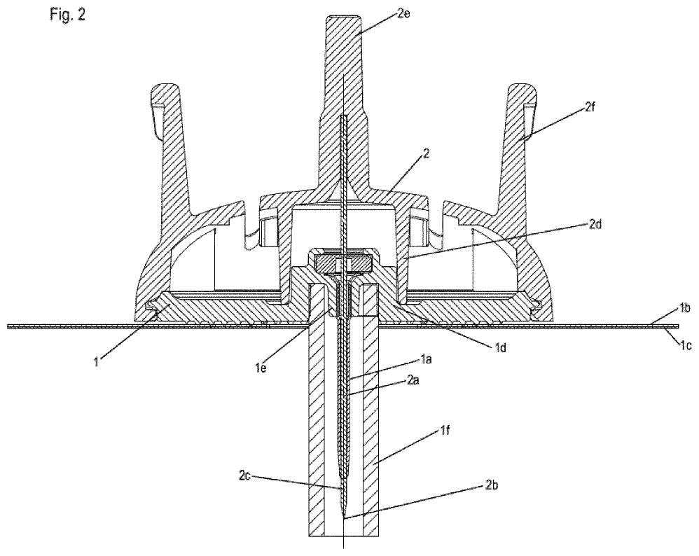

FIG. 2 shows a longitudinal sectional view of the infusion set according to FIG. 1, where the cap part (2) is connected, in particular snapped, to the base part (1).

The needle (2a) of the cap part (2) is received in the cannula (1a) of the base part (1) such that at least the distal tip of the needle (2b) of the needle (2a) of the cap part (2) projects beyond a distal end of the cannula (1a) of the base part (1). The concentric arrangement of the cannula (1a) and the needle (2a) serves to guide the cannula (1a) when the cannula (1a) of the base part (1) is inserted into the patient's skin. The protective element (1f) is detachably pressed into the base cylinder (1d), where the protective element (1f) is arranged between the base cylinder (1d) and the cannula cylinder (1e). The cap part 2 is snapped into the base part 1. For this purpose, a cap cylinder (2d) provided on the cap part (2) is in engagement with the base cylinder (1d) provided on the base part (1). Particularly, an anti-rotation device can be provided between the base part (1) and the cap part (2), such that the patient can more easily remove the protective film (1c) of the adhesive bandage (1b) from the base part (1).

To attach the infusion set to the patient's skin, the patient removes the protective element (1f) from the base part (1). The patient then removes the protective film (1c) from the adhesive bandage (1b). The cap part (2) snapped onto the base part (1) is placed on the patient's skin. The cannula (1a) of the base part (1) with the needle (2a) of the cap part (2) accommodated therein penetrates through the skin into the subcutaneous tissue. The cap part (2) is then removed from the base part (1) by the patient pre-tensioning the two holding elements (2f) inwards or pressing them towards each other, where the snap connection between the cap part (2) and the base part (1) is released. The needle (2a) is thus withdrawn from the subcutaneous tissue and the cannula (1a). The protective element (1f) can be placed over the needle (2a) provided on the cap part (3) in order to protect the patient from puncture injuries.

Afterwards, a connector part with a tube will be attached to the base part (1). The tube includes an adapter for detachable attachment to an infusion device, in particular to an infusion pump.

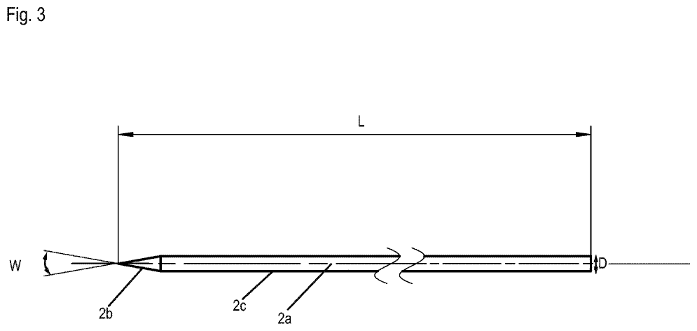

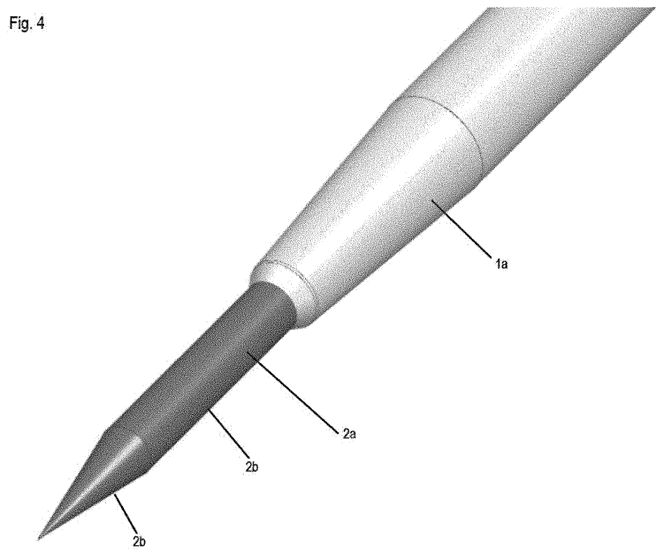

FIG. 3 shows a longitudinal sectional view of the needle (2a) of the cap part (2) according to FIG. 1. Furthermore, FIG. 4 shows an isometric view of the needle (2) of the cap part (2) according to FIG. 2, where the needle (2a) is arranged within the cannula (1a) of the base part (1) and at least the distal tip of the needle (2b) projects beyond the distal end of the cannula (1a).

The conical tip of the needle (2b) can be straight or oblique. Particularly, the conical tip of the needle (2b) is straight. The conical tip of the needle (2b) has a cone angle (W) greater than or equal to 10° and less than or equal to 35°, between 14° and 30°, such as approximately 20°, as shown in FIG. 3. This ensures that when the cannula (1a) is inserted into the patient's skin or subcutaneous tissue, the patient's skin or subcutaneous tissue is damaged as little as possible. In addition, when the needle is used according to implementations of the present disclosure, the patient's skin is penetrated in such a way that the skin or subcutaneous tissue heals as quickly and scar-free as possible after the puncture.

Furthermore, the length (L) of the needle (2a) projecting from the distal side of the base part (1) is greater than or equal to 10 mm and less than or equal to 40 mm, between 20 mm and 30 mm, such as approximately 24.4 mm. The length (L) of the needle (2a) is preferably measured from the distal side of the cannula cylinder (1d) of the base part (1) to the distal end of the tip of the needle (2b). These lengths (L) of the needle (2a) are particularly suitable for a cannula (1a) of approximately 5 mm to 10 mm, measured from the distal end of the cannula cylinder (le), so that at least the distal tip of the needle (2b) or the entire tip of the needle (2b) or at least part of the shaft of the needle (2c) projects beyond the distal end of the cannula (1a) when the needle (2b) is arranged within the cannula (1a), as shown in FIG. 4. This arrangement ensures that the cannula (1a) can be inserted into the patient's skin or subcutaneous tissue as painlessly as possible and without causing major injuries to the skin or subcutaneous tissue.

In addition, the diameter (D) of the needle (2a) is greater than or equal to 0.2 mm and less than or equal to 0.55 mm, between 0.26 mm and 0.5 mm, such as approximately 0.36 mm. The diameter (D) of the needle (2a) is measured on the straight shaft of the needle (2c). This diameter (D) is particularly suitable for a cannula (1a) with a diameter of 0.2 mm to 0.6 mm. The diameter (D) of the needle (2a) is matched to the diameter of the cannula (1a) so that the cannula can be guided well and, in particular, inserted into the patient's skin or subcutaneous tissue painlessly and without injury.

Embodiments of the present disclosure have been presented for the purpose of illustration and description. They are not intended to be exhaustive or to limiting. Obvious modifications or variations are possible in light of the above teachings. The embodiments were chosen and described to provide the best illustration of the principles of the implementations and the practical applications thereof, and to enable one of ordinary skill in the art to utilize the invention in various embodiments and with various modifications as are suited to the particular use contemplated.

### REFERENCE SIGNS

1 Base part
1a Cannula
1b Adhesive bandage
1c Protective film
1d Base cylinder
1e Cannula cylinder
1f Protective element
2 Cap part
2a Needle
2b Tip of the needle
2c Shaft of the needle
2d Cap cylinder
2e Grip part
2f Retaining element
L Length
D Diameter
W Cone angle

What is claimed is:

1. An infusion set, comprising:
a base part, wherein the base part comprises a proximal side and a distal side, wherein a cannula configured for supplying a liquid medicament into a patient is provided on the distal side of the base part;
a cap part configured for holding the infusion set by a user, by an insertion device or by a piercing aid, wherein the cap part comprises a proximal side and a distal side, and wherein a needle is provided on the distal side of the cap part,
wherein the base part and the cap part are configured to be connectable to each other, and when connected are configured for connecting the base part to skin of a patient,
wherein the needle is arranged within the cannula and at least a distal end of a tip of the needle projects beyond a distal end of the cannula, and
wherein the tip of the needle has a conical shape and the needle is made of a solid material.

2. The infusion set according to claim 1, wherein the tip of the needle is straight or obliquely conical.

3. The infusion set according to claim 1, wherein the cannula is a soft cannula.

4. The infusion set according to claim 1, wherein the base part and the cap part are connected in a rotationally-fixed manner.

5. The infusion set according to claim 1, wherein a diameter of the needle is greater than or equal to 0.2 mm and less than or equal to 0.55 mm.

6. The infusion set according to claim 5, wherein the diameter of the needle is between 0.26 mm and 0.5 mm.

7. The infusion set according to claim 6, wherein the diameter of the needle is about 0.36 mm.

8. The infusion set according to claim 1, wherein a length of the needle projecting from the distal side of the base part is greater than or equal to 10 mm and less than or equal to 40 mm.

9. The infusion set according to claim 8, wherein the length of the needle is between 20 mm and 30 mm.

10. The infusion set according to claim 9, wherein the length of the needle is about 24.4 mm.

11. The infusion set according to claim 1, wherein the tip of the needle is conical and has a cone angle greater than or equal to 10° and less than or equal to 35°.

12. The infusion set according to claim 11, wherein the cone angle of the conical tip of the needle is between 14° and 30°.

13. The infusion set according to claim 12, wherein the cone angle of the conical tip of the needle is about 20°.

14. The infusion set according to claim 1, wherein a diameter of the needle is greater than or equal to 0.2 mm and less than or equal to 0.55 mm, a length of the needle projecting from the distal side of the base part is greater than or equal to 10 mm and less than or equal to 40 mm, and the tip of the needle is conical and has a cone angle greater than or equal to 10° and less than or equal to 35°.

15. The infusion set according to claim 14, wherein the diameter of the needle is between 0.26 mm and 0.5 mm, the length of the needle is between 20 mm and 30 mm, and the cone angle of the conical tip of the needle is between 14° and 30°.

16. The infusion set according to claim 15, the diameter of the needle is about 0.36 mm, the length of the needle is about 24.4 mm, and the cone angle of the conical tip of the needle is about 20°.

17. The infusion set according to claim 1, wherein the needle is made of metal.

18. The infusion set according to claim 17, wherein the metal comprises stainless steel, stainless steel according to ISO15510, or a steel alloy.

19. The infusion set according to claim 1, wherein the needle is made of one or more of: iron, copper, bronze, aluminum, magnesium or an alloy of combinations thereof.
